(19) United States
(12) Patent Application Publication
(10) Pub. No.: US 2022/0323615 A1
(43) Pub. Date: Oct. 13, 2022

(54) SOLVATOCHROMIC INDICATOR FOR DETECTING INFECTION

(71) Applicant: Dasia TAYLOR, North Liberty, IA (US)

(72) Inventor: Dasia TAYLOR, North Liberty, IA (US)

(21) Appl. No.: 17/717,860

(22) Filed: Apr. 11, 2022

Related U.S. Application Data
(60) Provisional application No. 63/173,611, filed on Apr. 12, 2021.

(51) Int. Cl. A61K 49/00; A61B 5/145; A61B 5/00; A61L 17/00

(52) U.S. Cl. CPC A61K 49/006; A61B 5/14539; A61B 5/742; A61K 49/0063; A61L 17/005

(57) ABSTRACT

A solvatochromic indicator for detecting the presence of a bacterial infection is disclosed. An indicator consisting of Beta vulgaris and further optionally comprising Curcuma longa, isopropyl alcohol, and any combination thereof, undergoes a detectable color change in the presence of an infection. Further provided are filaments treated with the solvatochromic indicator wherein the detectable color change of the indicator is based on the pH of the tissues with a bacterial infection.

### RELATED APPLICATIONS

This application claims priority to U.S. Provisional Patent Application No. 63/173,611 filed Apr. 12, 2021, the content of which is incorporated herein by reference in its entirety.

### FIELD OF THE INVENTION

The present invention is directed towards a composition and methods for efficiently detecting changes in pH using solvatochromic indicators. In a specific embodiment, the present invention is also directed towards efficiently detecting infections at surgical sites based on the visual detection of pH changes.

### BACKGROUND

Surgical site infections are one of the most common causes of Nosocomial or hospital-acquired infections. These infections are commonly caused by gram positive bacterial species like Staphylococcus aureus and Streptococcus pyogenes as well gram negative bacteria such as Pseudomonas aeruginosa or Escherichia coli. Clinical symptoms can include localized redness, pain, wound drainage, delayed healing, swelling, and fever.

Millions of surgical procedures are performed at inpatient hospitals, ambulatory hospital-affiliated hospitals, or freestanding settings. Surgical site infections can occur in up to five percent of these surgical procedures and the infections are associated with significant morbidity and mortality. In addition, it often leads to an increased hospital stay and additional costs from prolonged antibiotic therapies.

The core guidelines for surgical procedures often include recommendations for the prevention of surgical site infections that are often generalized across surgical procedures. Among these recommendations, administering preoperative antimicrobial agents before a skin incision, cleaning the skin clean before and after surgery, and wound monitoring are among the most commonly used methods. In addition, hospitals around the world have adjusted their surgery protocols in an effort to reduce the likelihood of a surgical site infection. However, the protocol for checking the incision site for obvious signs of infections has not changed, despite the fact that once signs of infection occur, it is very difficult to get ahead, thus the reason why surgical site infections are so dangerous. If the surgical site infection is caught early, the wound can be treated with antibiotics as opposed to requiring additional surgery. These concerns are broadly applicable to any type of wound site—particularly in less developed countries where medical treatment can be difficult to obtain.

To this end, there exists a need for methods of efficiently detecting bacterial infections in both clinical and non-clinical environments. The composition, methods, and uses of the present disclosure provide for efficiently detecting pH changes in a medical, biomedical, veterinary, or clinical environment or any other environment with a need to be kept free of bacteria. Detection of pH changes can be applied to visually detect infections at wound sites such as surgical sites. Disclosed herein are novel methods for detecting pH changes and, by extension, bacterial infections in any environment.

Furthermore, there exists a need for cheap and easily accessible tools to diagnose infection in developing countries. In these countries, people might not have the ability to see a doctor, and usage of a simple and broadly applicable composition to indicate infection will allow someone to know that medical attention is needed.

### SUMMARY OF THE INVENTION

In an aspect, the present disclosure provides a composition comprising extract from at least one Beta vulgaris bulb, extract from at least one Curcuma longa root, and at least 10% alcohol.

In an aspect, the present disclosure provides a composition comprising a solvatochromic indicator, wherein the solvatochromic indicator is a pH responsive visual indicator, and the composition further comprises extract from at least one Curcuma longa root.

In an aspect, the present disclosure provides a method comprising preparing a first extract from at least one Beta vulgaris bulb, preparing a second extract from at least one Curcuma longa root, and combining the first and second extracts in a solution comprising alcohol.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present embodiments are illustrated by way of the figures of the accompanying drawings, which may not necessarily be to scale, in which like references indicate similar elements, and in which:

FIG. 1: FIG. 1A shows a color scale of Beta vulgaris extract over a range of pH conditions. FIG. 1B shows a set of polyester-cotton blend sutures treated with Beta vulgaris extract. From left to right, the sutures are subjected to a solution of pH 5, a no solution control, and a solution of pH 9. This image shows the sutures after 5 minutes has elapsed. FIG. 1C shows the same set of three sutures as seen in FIG. 1B except that approximately 60 hours has elapsed. The suture treated with Beta vulgaris extract and a solution of pH 9 is visually distinct from the control and the pH 5 treatments.

FIG. 2: FIG. 2A depicts a set of swabs. Each of the swabs has been treated with 1 ml of Beta vulgaris bulb extract. FIG. 2B depicts the same set of swabs one hour after application of the following solutions; from left to right: control with nothing added, pH 5 solution, pH 9 solution, distilled water, 91% alcohol, and a pH 9 solution followed by application of 1 ml of water.

FIG. 3: FIG. 3 demonstrates the antibacterial properties of a mixture comprising extract from a Beta vulgaris bulb. FIG. 3A shows the normal growth of E. coli when grown on a petri dish at 37° C. for 12 hours. FIG. 3B shows the same petri dish as FIG. 3A except that a filament comprising extract from a Beta vulgaris bulb is overlaid on this portion of the dish and no E. coli growth can be seen. FIG. 3C is a picture taken after removal of the filament and, again, no E. coli growth can be seen in or around the area on which the filament had touched.

FIG. 4: FIG. 4A is an exemplary illustration of the chemical structure of parylene C which can be used as a thin-film coating compound in conjunction with the solvatochromic indicator of the present invention. FIG. 4B is an exemplary illustration of the steps in the coating process for the thin-film coating for parylene C. (VSi Parylene “Coating and Deposition Process” https://vsiparylene.com/about-parylene/#deposition-process).

FIG. 5: FIGS. 5A-C show three sets of sutures, each set containing three sutures, comprising extract from Beta vulgaris and extract from Curcuma longa mixture in 10% alcohol and coated with parylene C. FIGS. 5D-F show the same three sets of sutures as shown in FIGS. 5A-C except FIG. 5D has been treated in a solution of pH 5 and FIG. 5F has been treated in a solution of pH 9. FIG. 5E does not have an added solution and represents a negative control. FIGS. 5G-I shows the same set of sutures as FIGS. 5D-F ten minutes after application of the solutions described above. The color change in response to the basic pH 9 is clear in comparison to FIGS. 5G-H.

FIG. 6: FIG. 6A shows two tubes of purple cabbage extract in a solution of pH 5 (left, labeled C5) and a solution of pH 9 (right, labeled C9). FIG. 6B shows two tubes of equal parts purple cabbage extract and Beta vulgaris extract in a solution of pH 5 (left, labeled BC5) and a solution of pH 9 (right, labeled BC9). FIG. 6C shows two tubes of Beta vulgaris extract in a solution of pH 5 (left, labeled B5) and a solution of pH 9 (right, labeled B9) shown as a positive control.

### DETAILED DESCRIPTION OF THE INVENTION

Unless defined otherwise, all technical and scientific terms used have the same meaning as commonly understood by one of ordinary skill in the art to which this disclosure belongs. Where a term is provided in the singular, the inventors also contemplates aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B— i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to be inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

As used herein, the singular form “a,” “an,” and “the” include plural references unless the context clearly dictates otherwise. For example, the term “a compound” or “at least one compound” may include a plurality of compounds, including mixtures thereof.

As used herein, betaine, also known as betaine anhydrous or trimethylglycine, is a neutral chemical compound with a positively charged cationic functional group. Betaine occurs naturally in the human body and can be found in foods such as beets and spinach. In an embodiment, betaine is obtained from at least one beet (Beta vulgaris). In a further embodiment, betaine is obtained from a source other than Beta vulgaris. In a further embodiment, betaine is used as a solvatochromic indicator at a wound site or surgical site to diagnose bacterial infection.

As used herein, pH is defined as a measure of acidity and alkalinity of a solution that is a number on a scale on which a value of 7 represents neutrality and lower numbers indicate increasing acidity and higher numbers increasing alkalinity and on which each unit of change represents a tenfold, or logarithmic, change in acidity or alkalinity. pH can also be defined as the negative logarithm of the effective hydrogen-ion concentration or hydrogen-ion activity in gram equivalents per liter of the solution. pH values below 7 are also known as acidic pH scores and pH values above 7 are also known as basic pH scores.

Healthy human skin is naturally acidic and typically has a pH of 5. Wound sites and surgical sites can become infected with microbes or bacteria, these infections potentially causing sickness or death in the patient. Infected wounds have a pH of 9 or higher. The present invention provides an apparatus and method for detecting infections at surgical sites using medical devices combined with a color-changing indicator.

As used herein, a solvatochromic indicator refers to, without being limited, a compound that can be used to access solvent polarity by means of UV, visible, or near-infrared spectroscopic measurements. See, e.g., Reichardt, Chem. Rev., 1994, pages 2319-2358. In an embodiment, a solvatochromic indicator provides a visual indication of a change in pH when exposed to a material with a basic pH relative to the previous environment of the solvatochromic indicator. In a further embodiment, a visual indication of a change in pH is a change in color of the composition comprising the solvatochromic indicator. In another embodiment, a solvatochromic indicator demonstrates a visual indication of a change in pH when applied to a wound site with a bacterial infection. In a further embodiment, a solvatochromic indicator comprising betaine darkens when exposed to a basic pH compared to a neutral pH which occurs when the indicator is applied to a site with a bacterial infection. In a further embodiment, a solvatochromic indicator comprising extract of a Beta vulgaris bulb darkens when exposed to a basic pH compared to a neutral pH which occurs when the indicator is applied to a site with a bacterial infection. In a further embodiment, a solvatochromic indicator comprising extract of a Beta vulgaris bulb changes from red to purple when exposed to a basic pH compared to a neutral or acidic pH which occurs when the indicator is applied to a site with a bacterial infection.

As used herein, “extract” refers to a composition, solid, liquid, or a combination of both, obtained from the breaking of cell walls and cell membranes. Molecules within cell walls and cell membranes can be extracted through any means commonly known in the art. In an embodiment, extract is prepared by routine processes such as blending, grinding, crushing, beating, rubbing, or any combination thereof, a material comprising molecules desired to be extracted. In an embodiment, extract is prepared via commonly practiced techniques in the art such as sonication. It is envisioned that any equivalent method known in the art can be used to prepare extract for use in the disclosed methods and compositions. Further processing of extract to remove impurities is also envisioned including but not limited to any type of size exclusion methods.

In an aspect, a solvatochromic pH indicator comprising extract from the bulb of a Beta vulgaris plant is used to dye sutures or wound coverings. Sutures or wound coverings that comprise a solvatochromic pH indicator comprising Beta vulgaris will change color in the presence of a basic pH. In a further aspect, a solvatochromic pH indicator marks a basic pH environment via a purple color. Bacterial infections are known to produce basic pH conditions at infected wound sites. In this way, the composition disclosed herein comprising a solvatochromic pH indicator comprising Beta vulgaris provides a novel method for detecting bacterial infection at a wound site.

The bulb of a Beta vulgaris plant contains the chemical betaine, which is a solvatochromic indicator. One example of a solvatochromic indicator containing betaine is Reichardt's dye. In an aspect, the indicator of the present invention undergoes a visible color change when the pH becomes more basic which occurs when bacteria are present and actively metabolizing. In a further aspect, the color change is a change to the color purple in a basic pH environment. In another aspect, a solvatochromic indicator of the present invention can contain Curcuma longa (turmeric) in the form of an extract obtained from the root of the plant. In a further aspect, Curcuma longa extract is mixed into a solution comprising a solvatochromic indicator. In another aspect, the Beta vulgaris is mixed in a solution with isopropyl, or any other alcohol. In a further aspect, a solution containing a solvatochromic indicator comprises about 1%, 2%, 3%, 4%, 5%, 6%, 7%, 8%, 9%, 10%, 11%, 12%, 13%, 14%, 15%, 16%, 17%, 18%, 19%, or at least 20% alcohol. In a further aspect, a solution containing a solvatochromic indicator comprises at least 13 percent alcohol. In a further aspect, a solution containing a solvatochromic indicator comprises at least 15 percent alcohol. In a further aspect, a solution containing a solvatochromic indicator comprises at least 20 percent alcohol. In a further aspect, a solution containing a solvatochromic indicator comprises at least 7 percent alcohol. In a further aspect, a solution containing a solvatochromic indicator comprises at least 5 percent alcohol. In a further aspect, a solution containing a solvatochromic indicator comprises at least 3 percent alcohol. In another aspect, alcohol is diluted in water in the final solution.

In an embodiment, a solvatochromic indicator comprises equal part Beta vulgaris extract and equal parts Curcuma longa extract. In an embodiment, a solvatochromic indicator comprises two parts Beta vulgaris extract and one part Curcuma longa extract. In an embodiment, a solvatochromic indicator comprises one part Beta vulgaris extract and two parts Curcuma longa extract. In an embodiment, a solvatochromic indicator comprises two parts Beta vulgaris extract and three parts Curcuma longa extract.

As used herein, filament refers to, without being limited, any material, natural or synthetic, suitable for absorption or coating of a composition comprising a solvatochromic indicator. In an aspect, the solvatochromic indicator comprises betaine. In a preferred embodiment, betaine is present in extract from a Beta vulgaris bulb. In one embodiment, a filament can be porous or non-porous. In a further embodiment, a filament is suitable for use in medical procedures. In a further embodiment, a filament is suitable for use as a wound suture. In another embodiment, a filament is suitable for use as a wound covering. Non-limiting examples of a filament includes gauze, medical swabs, cotton, prolene, silk, polyester, catgut, polyglactin, polyglycol, poliglecaprone, polydioxanone, polypropylene, polyamide, and mixtures thereof. In a further embodiment, a filament is composed of synthetic fiber. In a further embodiment, a filament is composed of natural fiber.

In an embodiment, filaments, such as sutures or wound coverings, may be saturated, infused, coated by, or otherwise treated with a solvatochromic indicator of the present invention. In an aspect, the solvatochromic indicator comprises betaine. In a preferred embodiment, betaine is present in extract from a Beta vulgaris bulb. In a further aspect, a method is provided by which the solvatochromic indicator comprising solution is added to the sutures or wound covering prior to application to a wound. In another aspect, a method is provided by which the solvatochromic indicator comprising solution is added to the sutures or wound covering after application to a wound. In each of these embodiments, the color change produced by the pH change triggered by the presence of bacteria is noticeable enough to be detected by an un-assisted human eye.

As used herein, a wound site is a location on a body, human or animal, in which the surface of the skin has been broken or abraded. In an aspect, a surgical site is a wound site. Without being limiting, a wound may be a laceration, cut, gash, tear, hole, incision, or lesion. In a further aspect, a wound site may be any environment prone to bacterial growth and/or infection.

As used herein, surgical site is defined in this disclosure as a particular part of the body, either human or animal, where surgery has taken place. A surgical site may also include sites on the body where other open wounds such as lacerations, cuts, gashes, tears, holes, incisions, or lesions have occurred. Surgical site infections may occur at any of these sites and may take a long time to detect using traditional methods.

Bacteria often associated with wound site infections include, in a non-limiting example, Staphylococcus sub species (ssp.), Streptococcus ssp., Pseudomonas ssp., Escherichia ssp., Enterococcus ssp., Proteus ssp., Serratia ssp., Enterobacter ssp., Acetinobacter ssp., and Klebsiella ssp. Further bacteria often associated with wound site infections, include, but are not limited to, Streptococcus pyogenes, Pseudomonas aeruginosa, Enterococcus faecalis, Proteus mirabilis, Serratia marcescens, Enterobacter cloacae, Acetinobacter anitratus, Klebsiella pneumoniae, Escherichia coli, Staphyloccus aureus, and coagulase-negative Staphylococci.

As used herein, a suture refers to a medical device that is commonly used to close incisions, punctures, lacerations, or the like, such as those that are present at surgical or wound sites, by holding body tissues together after surgery. A suture is typically a thread connected to a needle that is stitched through the open body tissues. The suture is secured using a surgical knot. In an embodiment, sutures are saturated with the solvatochromic indicator of the current disclosure. In another embodiment, sutures are coated with the solvatochromic indicator of the current disclosure. In an aspect, the solvatochromic indicator comprises betaine. In a preferred embodiment, betaine is present in extract from a Beta vulgaris bulb.

Wound coverings are commonly used to enclose or encapsulate a wound or surgical site. Non-limiting examples of wound coverings include gauze sponge, gauze bandages, non-adherent pads, non-adherent wound dressings, foam dressings, calcium alginates, hydrogel dressings, hydrocolloids, alginates, composites, and transparent dressings. In one embodiment, any solvatochromic indicator of the present disclosure can be used in conjunction with any wound covering. In an aspect, the solvatochromic indicator comprises betaine. In a preferred embodiment, betaine is present in extract from a Beta vulgaris bulb.

As used herein, a thin-film coating refers to deposition of a substance over a material comprising any solvatochromic indicator of the present disclosure. In an aspect, the solvatochromic indicator comprises betaine. In a preferred embodiment, betaine is present in extract from a Beta vulgaris bulb. In a further embodiment, a thin-film coating allows interaction of the outside environment with the solvatochromic indicator of the present disclosure. In an embodiment, a thin-film coating of the present invention is a thin-film coating of parylene C or any suitable substance with similar properties. A thin-film coating can be coated onto any material comprising any solvatochromic indicator of the present disclosure. A thin-film coating can be deposited onto any material comprising any solvatochromic indicator of the present disclosure.

In an aspect, the present disclosure provides a method comprising contacting a wound with a filament comprising at least one solvatochromic indicator, detecting a change in pH based on a visual change in that at least one solvatochromic indicator, and diagnosing a bacterial infection based on that visual change. In a further embodiment, the at least one solvatochromic indicator is extract from at least one Beta vulgaris bulb. In a further embodiment, the solution comprises extract from at least one Curcuma longa root. In a further embodiment, the solution comprises about 1%, 2%, 3%, 4%, 5%, 6%, 7%, 8%, 9%, 10%, 11%, 12%, 13%, 14%, 15%, 16%, 17%, 18%, 19%, or at least 20% alcohol. In an aspect, the solution further comprises at least 13 percent alcohol. In an aspect, the solution further comprises at least 15 percent alcohol. In an aspect, the solution further comprises at least 20 percent alcohol. In an aspect, the solution further comprises at least 7 percent alcohol. In an aspect, the solution further comprises at least 5 percent alcohol. In an aspect, the solution further comprises at least 3 percent alcohol. In another aspect, alcohol is diluted in water in the final solution. In another embodiment, the solvatochromic indicator is betaine. In another embodiment, the wound is contacted with a filament in the form of a suture. In another embodiment, the wound is contacted with a filament in the form of a wound covering. In a further embodiment, a filament is coated with a thin-film coating.

In an aspect, the present disclosure provides a method comprising contacting a wound with a filament, applying a solution comprising at least one solvatochromic indicator to that filament, detecting a change in pH based on a visual change in that at least one solvatochromic indicator, and diagnosing a bacterial infection based on that visual change. In a further embodiment, the at least one solvatochromic indicator is extract from at least one Beta vulgaris bulb. In a further embodiment, the solution comprises extract from at least one Curcuma longa root. In a further embodiment, the solution comprises 1%, 2%, 3%, 4%, 5%, 6%, 7%, 8%, 9%, 10%, 11%, 12%, 13%, 14%, 15%, 16%, 17%, 18%, 19%, or at least 20% alcohol. In an aspect, the solution further comprises at least 13 percent alcohol. In an aspect, the solution further comprises at least 15 percent alcohol. In an aspect, the solution further comprises at least 20 percent alcohol. In an aspect, the solution further comprises at least 7 percent alcohol. In an aspect, the solution further comprises at least 5 percent alcohol. In an aspect, the solution further comprises at least 3 percent alcohol. In another aspect, alcohol is diluted in water in the final solution. In another embodiment, the solvatochromic indicator is betaine. In another embodiment, the wound is contacted with a filament in the form of a suture. In another embodiment, the wound is contacted with a filament in the form of a wound covering. In a further embodiment, a filament is coated with a thin-film coating.

In an aspect, the present disclosure provides a method comprising preparing a first extract from at least one Beta vulgaris bulb, preparing a second extract from at least one Curcuma longa root, and combining said first and second extracts in a solution comprising alcohol. In an embodiment, the solution comprises about 1%, 2%, 3%, 4%, 5%, 6%, 7%, 8%, 9%, 10%, 11%, 12%, 13%, 14%, 15%, 16%, 17%, 18%, 19%, or at least 20% alcohol. In a further aspect, the solution further comprises at least 13 percent alcohol. In a further aspect, the solution further comprises at least 15 percent alcohol. In a further aspect, the solution further comprises at least 20 percent alcohol. In a further aspect, the solution further comprises at least 7 percent alcohol. In a further aspect, the solution further comprises at least 5 percent alcohol. In a further aspect, the solution further comprises at least 3 percent alcohol. In another aspect, alcohol is diluted in water in the final solution. In another embodiment, the method comprises apply the solution to a filament. In a further embodiment, a filament is in a form selected from the group consisting of a suture, swabs, gauze sponge, gauze bandages, non-adherent pads, and non-adherent wound dressings. In another embodiment, the method comprises enclosing the filament in a thin-film coating. In a further embodiment, a thin-film coating is a coating of parylene C. In a further embodiment, a first extract from at least one Beta vulgaris bulb and a second extract from at least one Curcuma longa root are prepared using sonication or an equivalent method to rupture the cell walls and membranes.

In an aspect, the present disclosure provides a composition for use in detecting a bacterial infection at wound sites comprising a solvatochromic indicator wherein said solvatochromic indicator is a pH responsive visual indicator. In another aspect, a solvatochromic indicator comprises extract from at least one Beta vulgaris bulb. In another embodiment, a solvatochromic indicator comprises extract from at least one Curcuma longa root. In a further embodiment, a solvatochromic indicator comprises extract from at least one Beta vulgaris bulb and extract from at least one Curcuma longa root. In an embodiment, a solvatochromic indicator comprises about 1%, 2%, 3%, 4%, 5%, 6%, 7%, 8%, 9%, 10%, 11%, 12%, 13%, 14%, 15%, 16%, 17%, 18%, 19%, or at least 20% alcohol. In an aspect, a solvatochromic indicator comprises at least 13 percent alcohol. In an aspect, a solvatochromic indicator comprises at least 15 percent alcohol. In an aspect, a solvatochromic indicator comprises at least 20 percent alcohol. In an aspect, a solvatochromic indicator comprises at least 7 percent alcohol. In an aspect, a solvatochromic indicator comprises at least 5 percent alcohol. In an aspect, a solvatochromic indicator comprises at least 3 percent alcohol. In another aspect, alcohol is diluted in water in the final solution. In another aspect, the alcohol is isopropyl alcohol.

In an aspect, the present disclosure provides a composition for use in detecting a bacterial infection at surgical sites comprising a solvatochromic indicator wherein said solvatochromic indicator is a pH responsive visual indicator. In another aspect, a solvatochromic indicator comprises betaine. In another embodiment, a solvatochromic indicator comprises extract from at least one Curcuma longa root. In a further embodiment, a solvatochromic indicator comprises betaine and extract from at least one Curcuma longa root. In another embodiment, a solvatochromic indicator comprises about 1%, 2%, 3%, 4%, 5%, 6%, 7%, 8%, 9%, 10%, 11%, 12%, 13%, 14%, 15%, 16%, 17%, 18%, 19%, or at least 20% alcohol. In an aspect, a solvatochromic indicator comprises at least 13 percent alcohol. In an aspect, a solvatochromic indicator comprises at least 15 percent alcohol. In an aspect, a solvatochromic indicator comprises at least 20 percent alcohol. In an aspect, a solvatochromic indicator comprises at least 7 percent alcohol. In an aspect, a solvatochromic indicator comprises at least 5 percent alcohol. In an aspect, a solvatochromic indicator comprises at least 3 percent alcohol. In another aspect, alcohol is diluted in water in the final solution. In another aspect, the alcohol is isopropyl alcohol.

In an aspect, the present disclosure provides a composition comprising a solvatochromic indicator and a filament. In an aspect, the solvatochromic indicator comprises betaine. In a preferred embodiment, betaine is present in extract from a Beta vulgaris bulb. In an embodiment, a filament is selected from the group consisting of cotton, prolene, silk, polyester, catgut, polyglactin, polyglycol, poliglecaprone, polydioxanone, polypropylene, and polyamide. In another embodiment, a filament comprises a thin-film coating. In a further embodiment, a thin-film coating comprises parylene C.

In an aspect, the present disclosure provides a composition comprising a solvatochromic indicator for use at or on a wound site. In an aspect, the solvatochromic indicator comprises betaine. In a preferred embodiment, betaine is present in extract from a Beta vulgaris bulb. In an embodiment, a wound site is on a human body. In another embodiment, a wound site in on an animal body. In a further embodiment, an animal is selected from the group consisting of dog, cat, cattle, sheep, goat, llama, pig, horse, and donkey.

It is appreciated by a person of ordinary skill in the art that the compositions and methods provided herein can be applied to applications not disclosed herein but related to any field in which the detection of bacterial growth is a known need. For example, compositions and methods provided herein can be adapted for use in the detection of bacteria growth on the surfaces of foodstuffs or preparation surfaces in the food industry. In an aspect, uses in the food industry are envisioned, in a non-limiting manner, in a professional kitchen, industrial kitchen, private kitchen, food storage facilities, food storage refrigerators, food storage freezers, etc. Additional applications are also envisioned in the biomedical industry where there is a known need for the detection of bacterial growth. For example, the use of catheters is common in many medical fields, i.e. nephrology, and there is a need for compositions and methods for the detection of bacteria. Catheters incorporating the disclosed compositions can meet these needs. Additionally, it is envisioned that additional agents may be added to the compositions of the instant application which provide for a binding affinity to materials that are not porous or otherwise demonstrate poor uptake of the disclosed solvatochromic indicators without said additional agents. In this manner, solid surfaces may be treated with a solvatochromic indicator and coated with a thin-film coating for use in the detection of bacterial growth as indicated by pH changes.

The following exemplary, non-limiting, embodiments are envisioned:

Embodiment 1: A composition comprising extract from at least one Beta vulgaris bulb, extract from at least one Curcuma longa root, and at least about 1%, 2%, 3%, 4%, 5%, 6%, 7%, 8%, 9%, 10%, 11%, 12%, 13%, 14%, 15%, 16%, 17%, 18%, 19%, or at least 20% alcohol.

Embodiment 2: The composition of Embodiment 1, further comprising a filament.

Embodiment 3: The composition of Embodiment 1 or 2, wherein the filament is in the form of a suture.

Embodiment 4: The composition of any one of Embodiments 1 to 3, further comprising a thin-film coating enclosing said filament.

Embodiment 5: The composition of any one of Embodiments 1 to 4, wherein the thin-film coating comprises parylene C.

Embodiment 6: A composition for use in detecting a bacterial infection at a wound site comprising a solvatochromic indicator wherein the solvatochromic indicator is a pH responsive visual indicator, wherein the composition further comprises extract from at least one Curcuma longa root.

Embodiment 7: The composition of Embodiment 6, wherein the solvatochromic indicator comprises betaine.

Embodiment 8: The composition of Embodiment 6 or 7, wherein the solvatochromic indicator comprises extract from at least one Beta vulgaris bulb.

Embodiment 9: The composition of any one of Embodiments 6 to 8, wherein the composition further comprises at least about 1%, 2%, 3%, 4%, 5%, 6%, 7%, 8%, 9%, 10%, 11%, 12%, 13%, 14%, 15%, 16%, 17%, 18%, 19%, or at least 20% alcohol.

Embodiment 10: The composition of any one of Embodiments 6 to 9, wherein the composition further comprises a filament.

Embodiment 11: The composition of any one of Embodiments 6 to 10, wherein the filament is in a form selected from the group consisting of a suture, gauze, gauze sponge, gauze bandages, non-adherent pads, and non-adherent wound dressings.

Embodiment 12: The composition of any one of Embodiments 6 to 11, further comprising a thin-film coating enclosing said filament.

Embodiment 13: The composition of any one of Embodiments 6 to 12, wherein the thin-film coating comprises parylene C.

Embodiment 14: A method comprising:

preparing a first extract from at least one Beta vulgaris bulb, preparing a second extract from at least one Curcuma longa root, and combining said first and second extracts in a solution comprising alcohol.

Embodiment 15: The method of Embodiment 14, further comprising application of the solution to a filament.

Embodiment 16: The method of Embodiment 14 or 15, further comprising enclosing the filament in a thin-film coating.

Embodiment 17: The method of any one of Embodiments 14 to 16, wherein the thin-film coating comprises parylene C.

Embodiment 18: The method of any one of Embodiments 14 to 17, wherein the filament is in a form selected from the group consisting of a suture, gauze sponge, gauze bandages, non-adherent pads, and non-adherent wound dressings.

Embodiment 19: The method of any one of Embodiments 14 to 18, wherein the first and the second extracts are prepared by sonication or an equivalent process.

Embodiment 20: The method of any one of Embodiments 14 to 19, wherein the solution comprises a concentration of at least about 1%, 2%, 3%, 4%, 5%, 6%, 7%, 8%, 9%, 10%, 11%, 12%, 13%, 14%, 15%, 16%, 17%, 18%, 19%, or at least 20% alcohol.

Embodiment 21: A composition for use in detecting a bacterial infection at wound sites comprising a solvatochromic indicator wherein the solvatochromic indicator comprises a pH responsive visual indicator.

Embodiment 22: A composition for use in detecting a bacterial infection at a wound site comprising extract from at least one Beta vulgaris bulb.

Embodiment 23: A composition for use in detecting pH changes at a wound site comprising extract from at least one Beta vulgaris bulb and extract from at least one Curcuma longa root.

Embodiment 24: A composition for use in detecting pH changes at a wound site comprising extract from at least one Beta vulgaris bulb, extract from at least one Curcuma longa root, and isopropyl alcohol.

Embodiment 25: A composition for use in detecting pH changes at a wound site comprising extract from at least one Beta vulgaris bulb, extract from at least one Curcuma longa root, and at least about 1%, 2%, 3%, 4%, 5%, 6%, 7%, 8%, 9%, 10%, 11%, 12%, 13%, 14%, 15%, 16%, 17%, 18%, 19%, or at least 20% alcohol.

Embodiment 26: A composition for use in detecting pH changes at a wound site comprising a filament, extract from at least one Beta vulgaris bulb, and extract from at least one Curcuma longa root.

Embodiment 27: A composition for use in detecting pH changes at a wound site comprising a filament, extract from at least one Beta vulgaris bulb, extract from at least one Curcuma longa root, and isopropyl alcohol.

Embodiment 28: A composition for use in detecting pH changes at a wound site comprising a filament, extract from at least one Beta vulgaris bulb, extract from at least one Curcuma longa root, and at least about 1%, 2%, 3%, 4%, 5%, 6%, 7%, 8%, 9%, 10%, 11%, 12%, 13%, 14%, 15%, 16%, 17%, 18%, 19%, or at least 20% alcohol.

Embodiment 29: A composition for use in detecting a bacterial infection at a wound site comprising betaine.

Embodiment 30: A composition for use in detecting a bacterial infection at a wound site comprising betaine and extract from at least one Curcuma longa root.

Embodiment 31: A composition for use in detecting pH changes at a wound site comprising a filament and betaine.

Embodiment 32: A composition for use in detecting pH changes at a wound site comprising a filament, betaine, and extract from at least one Curcuma longa root.

Embodiment 33: A composition comprising a filament, betaine, extract from at least one Curcuma longa root, and isopropyl alcohol for use in detecting pH changes at a wound site.

Embodiment 34: A composition comprising a filament, betaine, extract from at least one Curcuma longa root, and at least about 1%, 2%, 3%, 4%, 5%, 6%, 7%, 8%, 9%, 10%, 11%, 12%, 13%, 14%, 15%, 16%, 17%, 18%, 19%, or at least 20% alcohol for use in detecting pH changes at a wound site.

Embodiment 35: A composition comprising a filament, betaine, and extract from at least one Curcuma longa root for use in detecting pH changes at a wound site.

Embodiment 36: The composition of any one of embodiments 21 to 35, wherein the filament is selected from the group consisting of cotton, prolene, silk, polyester, catgut, polyglactin, polyglycol, poliglecaprone, polydioxanone, polypropylene, polyamide.

Embodiment 37: The composition of any one of embodiments 21 to 36, wherein a filament further comprises a thin-film coating.

Embodiment 38: The composition of any one of embodiments 21 to 37, wherein a thin-film coating comprises parylene C.

Embodiment 39: The composition of any one of embodiments 23 to 28, 30, and 32 to 35, wherein the extract from at least one Curcuma longa root comprises at least about 10%, 20%, 30%, 40%, 50%, or 60% of the composition.

Embodiment 40: The composition of any one of embodiments 22 to 28 and 32 to 35, wherein the extract from the bulb of at least one Beta vulgaris bulb comprises at least about 10%, 20%, 30%, 40%, 50%, or 60% of the composition.

Embodiment 41: The composition of any one of embodiments 1 to 13 and 21 to 40, wherein the composition is capable of detecting bacterial infection on a wound site on a human body.

Embodiment 42: The composition of any one of embodiments 1 to 13 and 21 to 40, wherein the composition is capable of detecting bacterial infection on a wound site on an animal body.

Embodiment 43: The composition of embodiment 42, wherein the animal is selected from the group consisting of dog, cat, cattle, sheep, goat, llama, pig, horse, and donkey.

Embodiment 44: A method comprising:

applying a filament comprising a solution comprising betaine, extract from at least one Curcuma longa root, and at least about 1%, 2%, 3%, 4%, 5%, 6%, 7%, 8%, 9%, 10%, 11%, 12%, 13%, 14%, 15%, 16%, 17%, 18%, 19%, or at least 20% alcohol to a wound site,

visually detecting a color change in the color indicator, and

diagnosing the presence or absence of an infection;

wherein the color change occurs when the color indicator is contacted with a basic pH.

Embodiment 45: A method comprising:

applying a wound covering to a wound site,

applying a solution comprising betaine to the wound covering,

visually detecting a color change in the color indicator, and

diagnosing the presence or absence of an infection;

wherein the color change occurs when the color indicator is contacted with a basic pH.

Embodiment 46: The method of embodiment 29 or 30, wherein the solution further comprises extract from at least one Curcuma longa root, and at least about 1%, 2%, 3%, 4%, 5%, 6%, 7%, 8%, 9%, 10%, 11%, 12%, 13%, 14%, 15%, 16%, 17%, 18%, 19%, or at least 20% alcohol.

Embodiment 47: The method of embodiment 27, wherein the infection is selected from the group consisting of Staphylococcus ssp., Streptococcus ssp., Pseudomonas ssp., Escherichia, ssp, Enterococcus ssp., Proteus ssp, Serratia ssp., Enterobacter ssp., Acetinobacter ssp., and Klebsiella ssp.

Embodiment 48: The method of any one of embodiments 14 to 20 and 44 to 47, wherein the method is performed on an animal.

Embodiment 49: The method of embodiment 48, wherein the animal is selected from the group consisting of dog, cat, cattle, sheep, goat, llama, pig, horse, and donkey.

Embodiment 50: The composition of any one of embodiments 1 to 13 and 21 to 40, wherein the composition is capable of use in a foodservice industry.

Embodiment 51: The composition of any one of embodiments 1 to 13 and 21 to 40, wherein the composition is applied to a catheter.

Embodiment 52: A composition comprising a solvatochromic indicator, wherein said solvatochromic indicator is a pH responsive visual indicator, wherein said composition further comprises extract from at least one Curcuma longa root.

Embodiment 53: The composition of embodiment 52, wherein said solvatochromic indicator is betaine.

Embodiment 54: The composition of embodiment 52 or 53, wherein said solvatochromic indicator is extract from at least one Beta vulgaris bulb.

Embodiment 55: The composition of any one of embodiments 52 to 54, wherein said composition further comprises at least about 1%, 2%, 3%, 4%, 5%, 6%, 7%, 8%, 9%, 10%, 11%, 12%, 13%, 14%, 15%, 16%, 17%, 18%, 19%, or at least 20% alcohol.

Embodiment 56: The composition of any one of embodiments 52 to 55, wherein said composition further comprises a filament.

Embodiment 57: The composition of any one of embodiments 52 to 56, wherein said filament is in a form selected from the group consisting of a suture, gauze, gauze sponge, gauze bandages, non-adherent pads, and non-adherent wound dressings.

Embodiment 58: The composition of any one of embodiments 52 to 57, further comprising a thin-film coating enclosing said filament.

Embodiment 59: The composition of any one of embodiments 52 to 58, wherein said thin-film coating comprises parylene C.

Having now generally described the disclosure, the same will be more readily understood through reference to the following examples that are provided by way of illustration, and are not intended to be limiting of the present disclosure, unless specified.

### EXAMPLES

### Example 1: Determining a Viable Material that would Change Colors with pH

The bulb of at least one Beta vulgaris (beet) plant is cut into small pieces measuring approximately 200 g. The cut pieces are added to 400 milliliters (ml) of distilled water in a beaker and boiled at 400° C. Filament material of a polyester-cotton blend is cut into two meter long strings and stirred into the boiling mixture and boiled for at least 30 minutes, stirring often. After boiling, the filaments are removed and allowed to dry on a non-absorbent surface. This process is repeated with different types of material including yarn, polyester, cotton, vicryl, and monocryl.

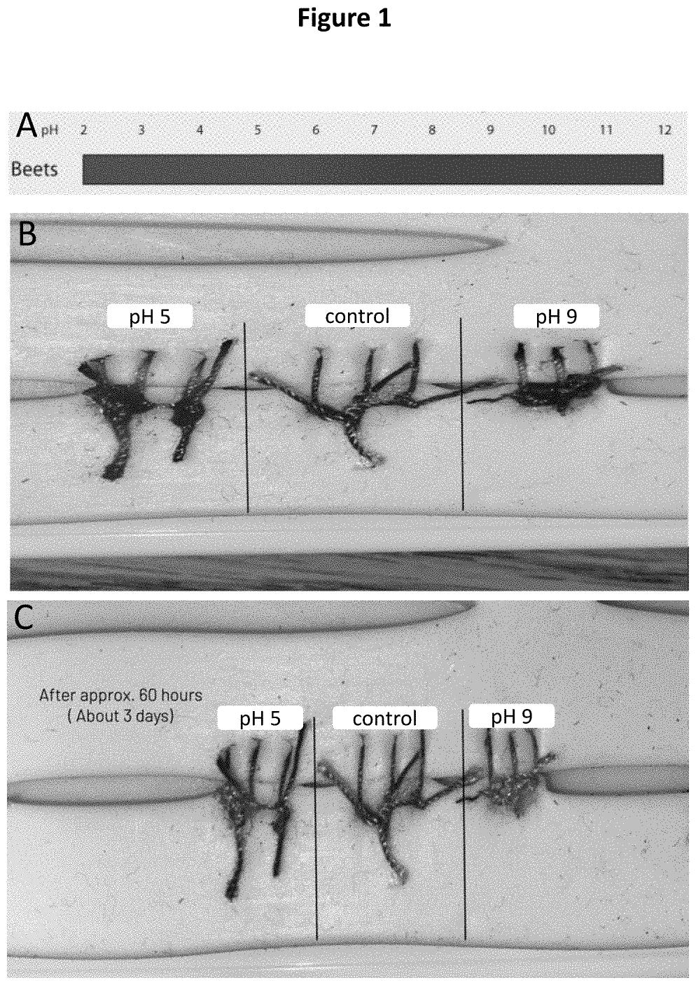

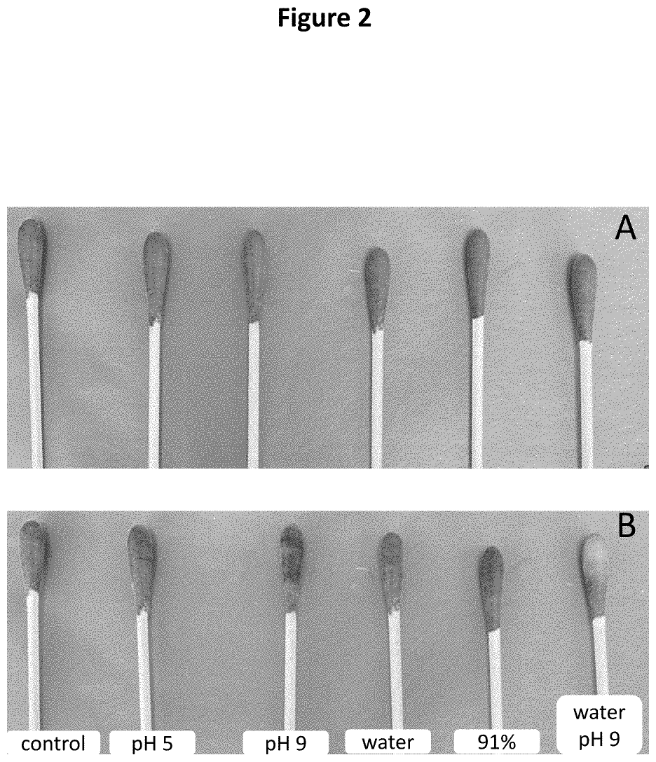

After drying, each of the filament materials is treated with a solution that is either pH 5, pH 7 (neutral, control), or pH 9 to determine strength of the visual response during the solvatochromic reaction. The solution appears red at pH 5 and purple at pH 9. See FIG. 1A. Materials treated with a solution of pH 9 demonstrated a color response clearly differentiating pH 9 from pH 5. The polyester-cotton blend demonstrated the most robust color reaction. See FIGS. 1B and C. Yarn (75% Viscose and 25% polyester) also demonstrated a robust color change at pH 9. The color change reaction of the Beta vulgaris extract is further tested by adding the extract to cotton swabs and testing against solutions at pH 5 and pH 9 as well as with the addition of water and 91% isopropyl alcohol. See FIG. 2. Addition of 91% isopropyl alcohol does not have any effect on the color change reaction and aids in the solubility of the extract containing solution. Superfluous water addition can reduce the color change reaction. See FIG. 2.

### Example 2: Production of Beta vulgaris Extract

Extract of Beta vulgaris is produced by cutting 250 grams of Beta vulgaris bulbs into small cubes. The Beta vulgaris cubes are added to 600 ml of distilled water and boiled at 400° C. for at least 45 minutes. The mixture is removed from heat and allowed to cool before being blended with an emersion blender. Alternatively, the mixture can be sonicated or the equivalent. The mixture is then put back onto heat and boiled for at least 30 minutes. The mixture is then removed from heat and cooled. 100 ml of 91% isopropyl alcohol is added and the mixture is funneled into a strainer to remove any remaining solid materials. The straining step can be repeated as needed until all solid material is removed.

### Example 3: Production of Curcuma longa Extract

Extract of Curcuma longa (turmeric) is produced by cutting 500 grams of turmeric root into small cubes. The Curcuma longa cubes are added to 1200 ml of distilled water and boiled at 400° C. for at least 45 minutes. The mixtures are removed from heat and allowed to cool before being blended with an emersion blender. Alternatively, the mixture can be sonicated or the equivalent. The mixture is then put back onto heat and boiled for at least 30 minutes. The mixture is then removed from heat and cooled. 100 ml of 91% isopropyl alcohol is added and the mixture is funneled into a strainer to remove any remaining solid materials. The straining step can be repeated as needed to until all solid material is removed.

### Example 4: Production of a Solvatochromic Mixture

Extract of Beta vulgaris and extract of Curcuma longa are prepared as described in Examples 2 and 3, respectively. A solvatochromic mixture comprising approximately 10% isopropyl alcohol is prepared by mixing equal parts extract of Beta vulgaris and extract of Curcuma longa. The solvatochromic mixture can be used in a manner similar to the extract of Beta vulgaris described in Example 1.

### Example 5: The Solvatochromic Mixture Comprises Anti-Bacterial Properties

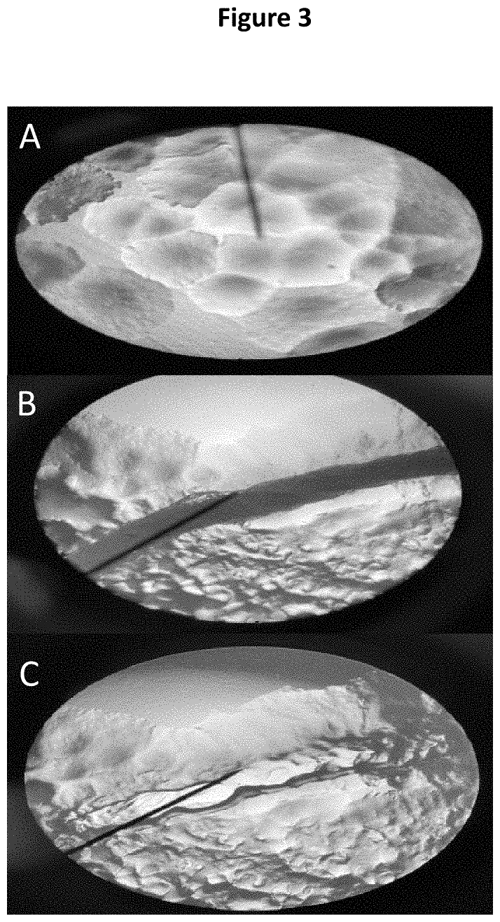

To examine whether the solvatochromic mixture of Examples 1 and 2 has anti-bacterial properties, extract of a Beta vulgaris bulb was exposed to E. Coli in culture. Petri dishes containing LB agar media, or similar, are made using standard procedures. The petri dishes are inoculated with E. Coli using standard laboratory procedures. Filament material is treated with the solvatochromic mixture of Examples 1, 2, 3, or 4 and boiled together to sterilize. After a brief cooling period, the filament material is placed on half of the petri plate, with the other half being a positive growth control. The plate is allowed to incubate at 37° C. for 12 hours. After incubation, the positive growth control side of the petri plate shows normal growth while the side treated with the solvatochromic mixture does not show growth confirming the anti-bacterial properties of the solvatochromic mixture. See FIG. 3.

### Example 6: Development of Materials Comprising a Solvatochromic Mixture

Sterile filaments in any form can be used to produce materials comprising solvatochromic indicators useful for diagnosing bacterial infections in biomedical environments or any other environment where bacterial growth is a concern, such as the food service industry.

For example, any materials suitable for sutures can be used to produce sutures comprising a solvatochromic mixture. Filament material is added to a tube with 35 ml of the solvatochromic mixture of Example 4. The material is allowed to soak in the solvatochromic mixture for 48 hours at room temperature. After 48 hours, the material is removed from the solvatochromic mixture and allowed to dry on a non-absorbent surface.

### Example 7: Thin-Film Coating of Treated Filaments

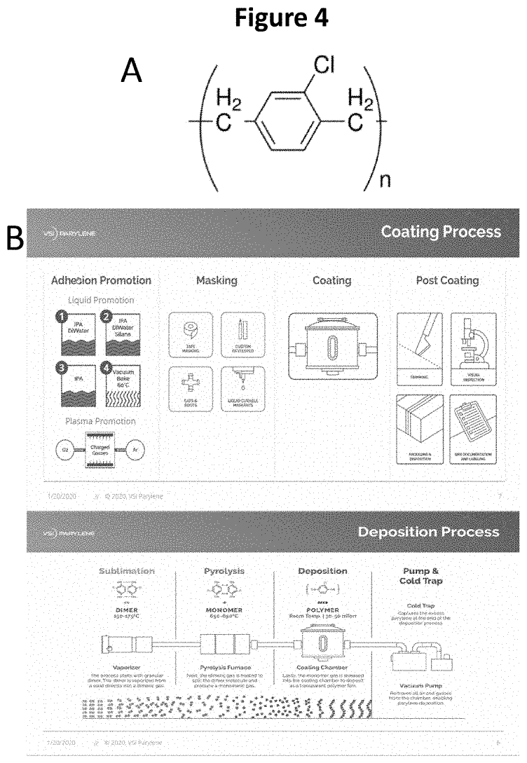

The materials produced comprising the solvatochromic mixture in Example 5 can be treated with a thin-film coating to lock in the solvatochromic mixture while allowing interaction with the outside environment. For Example, a thin-film coating of parylene C (FIG. 4A) is shown to work well with the solvatochromic mixture of the instant disclosure, locking in the solvatochromic mixture while being permeable enough to allow detection of the pH in an outside environment. Briefly, any deposition process can be used to apply a thin-film coating. Here, sutures treated with the solvatochromic mixture are coated using the VSi parylene C deposition process (FIG. 4B, VSi Parylene, Broomfield Colo., 80021). Parylene C is deposited using a liquid promotion adhesion protocol and subsequent coating as shown in FIG. 4B.

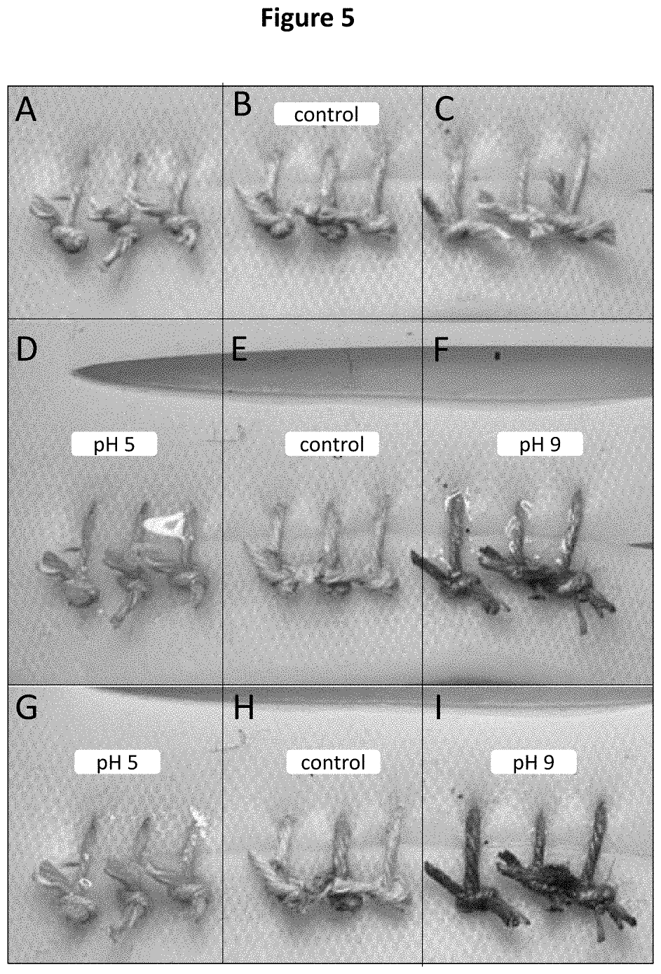

Parylene C treated sutures are stitched into a material mimicking skin. Three sets of sutures are set up, one to be treated with a pH 5 solution, one to be treated with a pH 9 solution, and one control. See FIG. 5A to C. The appropriate pH solutions are added to the stitches as timepoint zero and the control is not treated. See FIG. 5D to F. A color change reaction can be seen in response to the pH 9 solution after 10 minutes while the pH 5 solution does not invoke a color change (compare FIG. 5G and 5I). It is noted that parylene C still allows absorption of the pH 9 solution that has diffused underneath the artificial membrane demonstrating that the thin-film coating does not affect uptake into the suture and subsequent color change (See subtle color change in the control stiches, FIG. 5H).

### Example 8: Identification of Additional Solvatochromic Indicators Useful for Diagnosing Bacterial Infection

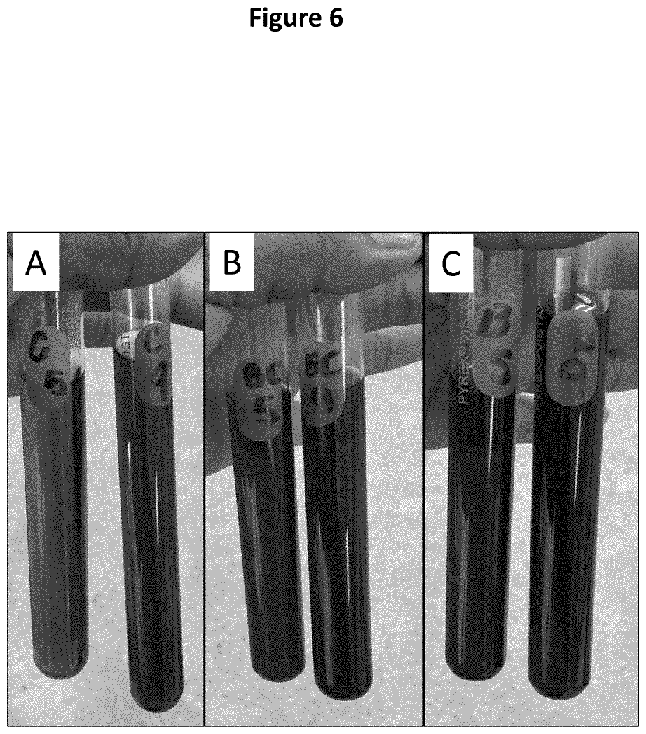

Any substance with solvatochromic properties allowing a visible color change reaction in response to a basic pH environment can be used to create solvatochromic indicator for use in the methods disclosed herein. For example, purple cabbage (Brassica oleracea with purple or red leaves), also known as red cabbage or Blaukraut, has suitable properties and is shown to elicit a strong color change in response to a pH 9 environment. See FIG. 6A

250 grams of purple cabbage leaves are cut into small strips and added to 600 ml of 400° C. distilled water and allowed to boil for at least 45 minutes. The strips are removed from heat and allowed to cool for 10 minutes. After the cooling period, an emersion blender is used to break down cell membranes. Sonication or any equivalent procedure can also be used to produce purple cabbage leaf extract. The mixture is then returned to heat and boiled for an additional 30 minutes. After cooling for at least 10 minutes, 100 ml of 91% isopropyl alcohol is added to the solution. The mixture is funneled into a strainer to remove any remaining solid materials. The straining step can be repeated as needed until all solid material is removed. The cabbage extract and be used alone or combined with Beta vulgaris extract. See FIGS. 6A and 6B.

1. A composition comprising extract from at least one Beta vulgaris bulb, extract from at least one Curcuma longa root, and at least 10% alcohol.

2. The composition of claim 1, further comprising a filament.

3. The composition of claim 2, wherein said filament is in the form of a suture.

4. The composition of claim 2, further comprising a thin-film coating enclosing said filament.

5. The composition of claim 4, wherein said thin-film coating comprises parylene C.

6. A composition comprising a solvatochromic indicator, wherein said solvatochromic indicator is a pH responsive visual indicator, wherein said composition further comprises extract from at least one Curcuma longa root.

7. The composition of claim 6, wherein said solvatochromic indicator comprises betaine.

8. The composition of claim 6, wherein said solvatochromic indicator comprises extract from at least one Beta vulgaris bulb.

9. The composition of claim 6, wherein said composition further comprises at least 7% alcohol.

10. The composition of claim 6, wherein said composition further comprises a filament.

11. The composition of claim 10, wherein said filament is in a form selected from the group consisting of a suture, gauze, gauze sponge, gauze bandages, non-adherent pads, and non-adherent wound dressings.

12. The composition of claim 10, further comprising a thin-film coating enclosing said filament.

13. The composition of claim 12, wherein said thin-film coating comprises parylene C.

14. A method comprising:
preparing a first extract from at least one Beta vulgaris bulb,
preparing a second extract from at least one Curcuma longa root, and
combining said first and second extracts in a solution comprising alcohol.

15. The method of claim 14, further comprising application of said solution to a filament.

16. The method of claim 15, further comprising enclosing said filament in a thin-film coating.

17. The method of claim 16, wherein said thin-film coating comprises parylene C.

18. The method of claim 15, wherein said filament is in a form selected from the group consisting of a suture, gauze sponge, gauze bandages, non-adherent pads, and non-adherent wound dressings.

19. The method of claim 15, wherein said first and said second extracts are prepared by sonication or an equivalent process.

20. The method of claim 14, wherein said solution comprises a concentration of at least 7% alcohol.
